(19) United States
(12) Patent Application Publication
(10) Pub. No.: US 2006/0089709 A1
(43) Pub. Date: Apr. 27, 2006

(54) Medical implant with average surface charge density

(76) Inventor: Michael N. Helmus, Worcester, MA (US)

Correspondence Address: JONES DAY, 222 EAST 41ST ST, NEW YORK, NY, 10017, US

(21) Appl. No.: 10/970,264

(22) Filed: Oct. 21, 2004

(51) Int. Cl. A61F 2/06

(52) U.S. Cl. 623/1.44

(57) ABSTRACT

A medical device for implantation into a host organism is disclosed. The device comprises a surface adapted for contact with body tissue of the host organism and an electrode disposed on at least a portion of the surface. Also the device comprises a power source in direct or indirect electrical communication with the electrode. The power source is capable of providing a current to the electrode to create an average surface charge density on the surface that is effective to promote the biocompatibility of the surface with the body tissue or create other desired biological effects.

### FIELD OF THE INVENTION

The present invention relates to medical devices that are implantable into host organisms. More specifically, the invention relates to medical devices having a charged surface for promoting certain biological effects. In particular, the charged surface of the device can promote biocompatibility of the medical device with the host organism and/or a biological effect, such as desired cell growth, at or near the site of implant. Also, the charged surface of the device in certain circumstances can promote thrombus formation, enhance inflammation or enhance tissue formation.

### BACKGROUND OF THE INVENTION

When an implant, such as a medical device, is inserted or placed into a host organism, the host organism's defense mechanisms may react to the implant in ways that reduce the effectiveness of the implant or result in adverse reactions in the host organism, e.g. inflammatory reaction in tissue surrounding the implant. Implants that do not harm the organism and do not provoke an adverse reaction to the implant are said to be more biocompatible than implants that harm the organism or provoke a significant adverse reaction to the implant.

In order to provide the surfaces of implants or medical devices with greater biocompatibility, coatings have been placed on the surfaces. For example, a variety of medical conditions have been treated by introducing an insertable medical device having a coating for release of a biologically active material. For example, various types of biologically active material-coated medical devices, such as stents, have been proposed for localized delivery of the biologically active material to a body lumen. See, e.g., U.S. Pat. No. 6,099,562 to Ding et al.

However, exposure to a medical device which is implanted or inserted into the body of a patient can cause the body tissue to exhibit adverse physiological reactions. For instance, the insertion or implantation of certain catheters or stents can lead to undesired coagulation or platelet aggregation leading to the formation of thrombus, clots or emboli in blood vessels. Other adverse reactions to vascular intervention includes smooth muscle cell proliferation which can lead to hyperplasia, restenosis, e.g. the re-occlusion of the artery or occlusion of blood vessels, and/or calcification. Restenosis is caused by an accumulation of extra cellular matrix containing collagen and proteoglycans in association with smooth muscle cells which is found in both the atheroma and the arterial hyperplastic lesion after balloon injury or clinical angioplasty. Treatment of restenosis often involves a second angioplasty or bypass surgery. The drawbacks of such treatment, including the risk of repeat restenosis, are obvious.

Furthermore, the effect of the surface of materials used to coat implants has been investigated. As discussed in Helmus et al.'s “The Effect of Surface Charge on Arterial Thrombosis”, J. of Biomedical Materials Research, vol. 18, pp. 165-183 (1984), the effect of the ionization of polymers on the amount of thrombus formed was studied. It was found that the amount of thrombus formed on the surface of implants of random copolymers of (L-glutamic acid co-L-leucine) implanted in the femoral and carotid arteries of dogs was related to the composition and degree of ionization. When the initial surface concentration of unionized glutamic acid is greater than 10%, the surface of the implants was completely covered with thrombus. For surface concentrations of unionized glutamic acid less than 10%, the amount of thrombus was a linear function of the degree of ionization. When 10% of the total surface sites consisted of ionized glutamic acid residues, there was no thrombus and only formed elements adhered to the surface.

Therefore, while coatings on the surfaces of implants or medical devices can increase the biocompatibility of the surfaces, there remains a need for other ways to increase the biocompatibility of the surfaces of medical devices or implants. Also, there is a need for ways to achieve other desired biological effects. For instance, in certain situations it may be desirable to promote thrombus formation, enhance inflammation or enhance tissue formation, such as fibrous tissue formation.

### SUMMARY OF THE INVENTION

In one embodiment, the present invention is directed to a medical device such as a stent that has a biocompatible surface. The biocompatibility of the surface is achieved or enhanced by creating an average surface charge density on the surface of the device that is effective to promote the biocompatibility of the surface. In one embodiment of the present invention, the medical device comprises a surface adapted for contact with body tissue of a host organism and an electrode disposed on at least a portion of the surface. The medical device also comprises a power source that is in direct or indirect electrical communication with the electrode. The power source is capable of providing a current to the electrode to create an average surface charge density on the surface that is effective to promote the biocompatibility of the surface with the body tissue.

Furthermore, in this embodiment, the average surface charge density can comprise a net negative or net positive charge of positive and negative charges. Moreover, the electrode can be less than about 150 nm in length and/or 150 nm in width. The medical device can comprise a substantially cylindrical shape, wherein the surface defines a boundary of the cylindrical shape, such as a stent. Additionally, the power source can comprise an induction coil, a battery or a pick-up coil. When the power source comprises an induction coil, such coil is capable of being tuned to a pre-selected frequency. Also, when the power source comprises an induction coil, the induction coil can be in communication with a remote generator capable of generating an oscillating magnetic field at the pre-selected frequency and the oscillating magnetic field is capable of creating a voltage across the induction coil. In addition, the average surface charge density that is created can be maintained by a direct current or an alternating current or an alternating current offset by a direct current baseline. Also, the average surface charge density can be greater than 5 μC/cm², preferably, the average surface charge density ranges from about 0.05 to about 500 μC/cm²; more preferably about 0.5 to about 50 μC/cm².

Another embodiment of the present invention is directed to a medical device for implantation into a host organism that comprises a first surface adapted for contact with a surface of a body lumen of the host organism. The body lumen contains a fluid. The device also comprises a second surface adapted for contact with the fluid contained in the body lumen. In addition, the device comprises an electrode disposed on at least a portion of the first or second surface of the device. Also, the device comprises a power source in direct or indirect electrical communication with the electrode. The power source is capable of providing a current to the electrode to create an average surface charge density on the first or second surface that is effective to promote the biocompatibility of the first or second surface with the surface of the body lumen or the fluid.

Moreover, in this embodiment, the average surface charge density can comprise a net negative or net positive charge of positive and negative charges. Also, the electrode can be less than about 150 nm in length and/or width. Also, the device can further comprise a controller disposed on the first or second surface of the device. The controller is in electrical communication with the power source and the electrode and the controller is capable of controlling the current provided to the electrode. Also, the electrode can be disposed on the first surface of the device and the average surface charge density is created on the first surface to promote the biocompatibility of the first surface with the surface of the body lumen. In addition, the electrode can be disposed on the second surface and the average surface charge density is created on the second surface to promote the biocompatibility of the second surface with the fluid contained in the body lumen. In some instances, the medical device can be a stent. In such instances, the first surface is an outer surface of the stent and the second surface is an inner surface of the stent. Additionally, the average surface charge density can be greater than 5 μC/cm². Preferably, the average surface charge density is in the range of about 0.05 to about 500 μC/cm². More preferably, the average surface charge density is in the range of about 0.5 to about 50 μC/cm². Also, the average surface charge density can range from about 3×10¹² to about 3×10¹⁴ charges /cm². Moreover, the power source can comprise a battery, a pick-up coil or an induction coil. When the power source comprises a pick-up coil, the pick-up coil can be disposed on the first surface of the device. Also, when the power source comprises a pick-up coil, such coil can be inductively coupled to a primary coil that is located external to the host organism.

In yet another embodiment, the invention is directed to a stent comprising a surface adapted for contact with the body tissue of a host organism. An electrode is disposed on at least a portion of the surface of the device. Also, the device comprises a power source comprising an induction coil that is in direct or indirect electrical communication with the electrode. The induction coil is capable of providing a current to the electrode to create an average surface charge density on the surface that is effective to promote the biocompatibility of the surface with the body tissue. The average surface charge density that is created is greater than 5 μC/cm² and comprises a net negative charge of positive and negative charges.

In another embodiment, the invention is directed to a medical device for implantation into a host organism in which the device comprises a surface adapted for contact with body tissue of the host organism. An electrode is disposed on at least a portion of the surface; and a power source in direct or indirect electrical communication with the electrode. The power source is capable of providing a current to the electrode to create an average surface charge density on the surface that is effective to produce a desired biological effect such as to result in blood coagulation, promote cell growth, promote thrombus formation, enhancing inflammation or enhancing tissue formation, such as fibrous tissue formation. These effects may be controlled, inter alia, by using a uniform positive or negative charge, a heterogenous mix of positively and negatively charged electrodes, the spatial distribution of the charges and/or the total net charge.

Also, in this embodiment, the tissue whose formation is enhance may be fibrous tissue. Furthermore, the average surface charge density can comprise a net positive charge of positive and negative charges. Moreover, the electrode can be less than about 150 nm in length and/or width. The medical device can comprise a substantially cylindrical shape, wherein the surface defines a boundary of the cylindrical shape, such as a stent. Additionally, the power source can comprise an induction coil, a battery or a pick-up coil. When the power source comprises an induction coil, such coil is capable of being tuned to a pre-selected frequency. Also, when the power source comprises an induction coil, the induction coil can be in communication with a remote generator capable of generating an oscillating magnetic field at the pre-selected frequency and the oscillating magnetic field is capable of creating a voltage across the induction coil. In addition, the average surface charge density that is created can be maintained by a direct current or an alternating current or an alternating current offset by a direct current baseline. Also, the average surface charge density can be greater than 5 μC/cm², preferably, the average surface charge density ranges from about 0.05 to about 500 μC/cm²; more preferably about 0.5 to about 50 μC/cm².

In yet another embodiment, the invention is directed to a method of promoting the biocompatibility of a medical device for implantation into a host organism. The method comprises obtaining a medical device having a surface adapted for contact with body tissue of the host organism. An electrode is disposed on at least a portion of the surface. Also, a power source is disposed in direct or indirect electrical communication with the electrode. The power source is capable of providing a current to the electrode to create an average surface charge density on the surface that is effective to promote the biocompatibility of the surface with the body tissue.

### BRIEF DESCRIPTION OF THE DRAWINGS

The invention will be described by reference to the preferred and alternative embodiments thereof in conjunction with the drawings in which:

FIG. 1 is a side view illustrating one embodiment of the present invention;

FIG. 2 is a magnified view of a portion of the embodiment shown in FIG. 1;

FIG. 3a is a cross-sectional view of a portion of a medical device of the present invention; and

FIG. 3b is a cross-sectional view of a portion of a medical device of the present invention.

### DETAILED DESCRIPTION

In one embodiment, the present invention is directed to a medical device having a biocompatible surface. The biocompatibility of the surface is achieved or enhanced by providing to or creating on a surface of a medical device an average surface an average charge density. Such an average surface charge density is provided to the surface by at least one electrode that is disposed on the surface. Preferably, a plurality of electrodes are employed. Also, preferably the electrodes are similar in size to cell receptors, such as less than 150 nm in length and/or width. The electrode is in electrical communication with a power source, such as a battery. The power source provides a current to the electrode which provides the surface on which the electrodes are disposed with an average surface charge density. The average surface charge density is the average of both positive and negative charges. (See Rosen J J. Gibbons, D F, Culp L A, “Fibrous Capsule Formation and Fibroblast Interactions at Charged Hydrogel Interfaces” In: Hydrogels Medical and Related Application, ed. J. D. Andrade ACS Symposium Series, Vol. 31, 1976, pp. 329-343.) Preferably to promote biocompatibility, the net average charge density should be negative.

The average surface charge density of a surface on which the electrodes are disposed is the total charges generated by the electrodes divided by the surface area on which the electrodes are disposed. To obtain the charge from a given electrode, the charge density of a particular electrode is multiplied by the surface area of that electrode. To obtain the total charges of all electrodes, the charge of each individual electrode is totaled. In the case where the medical device is a stent, the surface charge density can be obtained for the blood contacting surface or the tissue contacting surface. Moreover, the average charge density can vary from one portion of the device to another.

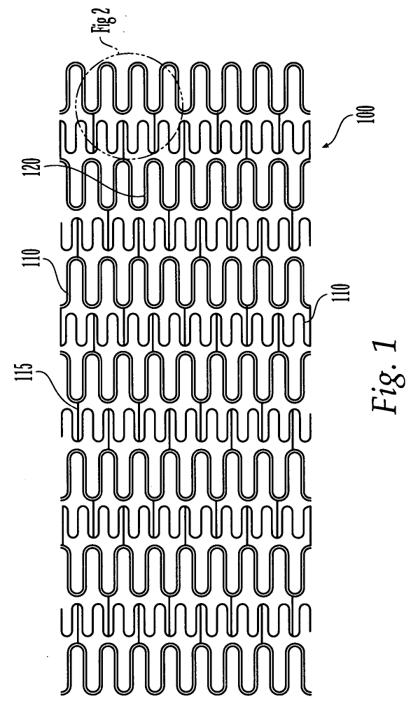

FIG. 1 sets forth one embodiment of the present invention. In this embodiment, the medical device is a stent 100 comprising a plurality of struts or circumferential members 110 that allow for expansion of the stent in the radial direction. The stent 100 shown in FIG. 1 has generally a cylindrical shape and may be implanted within a tubular organ or body lumen such as for example, an artery, or a duct. The particular mechanical design of the stent shown in FIG. 1 is for illustrative purposes and it should be understood that other stent designs may be used in, and are encompassed by, the present invention. Also, other medical devices in addition to stents can be used in the present invention.

The medical devices suitable for the present invention include, but are not limited to, stents, surgical staples, catheters, such as central venous catheters and arterial catheters, guide wires, cannulas, cardiac pacemaker leads or lead tips, cardiac defibrillator leads or lead tips, implantable vascular access ports, vascular or other grafts, intra-aortic balloon pumps, heart valves, cardiovascular sutures, total artificial hearts and ventricular assist pumps.

Medical devices which are particularly suitable for the present invention include any kind of stent for medical purposes, which is known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 4,733,665 issued to Palmaz, U.S. Pat. No. 4,800,882 issued to Gianturco, U.S. Pat. No. 4,886,062 issued to Wiktor and U.S. Pat. No. 5,449,373 issued to Pinchasik et al. A bifurcated stent is also included among the medical devices suitable for the present invention.

The medical devices suitable for the present invention may be fabricated from polymeric, ceramic and/or metallic materials. Examples of such polymeric materials include polyurethane and its copolymers, silicone and its copolymers, ethylene vinyl-acetate, poly(ethylene terephthalate), thermoplastic elastomer, polyvinyl chloride, polyolephines, cellulosics, polyamides, polyesters, polysulfones, polytetrafluoroethylenes, acrylonitrile butadiene styrene copolymers, acrylics, polyactic acid, polyclycolic acid, polycaprolactone, polyacetal, poly(lactic acid), polylactic acid-polyethylene oxide copolymers, polycarbonate cellulose, collagen and chitins. Examples of suitable metallic materials include metals and alloys based on titanium (e.g., nitinol, nickel titanium alloys, thermo-memory alloy materials), stainless steel, platinum, tantalum, nickel-chrome, certain cobalt alloys including cobalt-chromium-nickel alloys (e.g., Elgiloy7 and Phynox7) and gold/platinum alloy. Metallic materials also include clad composite filaments, such as those disclosed in WO 94/16646.

Furthermore, the surface area between electrodes, regardless of whether the surface is metal, ceramic or polymer, can be modified by coating the surface with a coating. For instance, to improve biocompatibility the surface can be coated or grafted with hydrogels, e.g., grafted PEG molecules or grafted bioactive molecules, e.g., heparin. The electrodes should be masked during the process of coating or grafting. Suitable coatings can comprise a polymer and/or a therapeutic agent.

Suitable polymers can be biostable or bioabsorbable. Preferably, the polymeric material is biostable. Preferably, the polymeric materials used in the coating compositions of the present invention are selected from the following: polyurethanes, silicones (e.g., polysiloxanes and substituted polysiloxanes), and polyesters. Also preferable as a polymeric material are styrene-isobutylene copolymers. Other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as poly(lactide-co-glycolide) (PLGA), polyvinyl alcohol (PVA), poly(L-lactide) (PLLA), polyanhydrides, polyphosphazenes, polycaprolactone (PCL), polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid (PLA), polyglycolic acid (PGA), polyethylene oxide (PEO), polylactic acid-polyethylene oxide copolymers, EPDM (etylene-propylene-diene) rubbers, fluorosilicones, polyethylene glycol (PEG), polyalkylene glycol (PAG), polysaccharides, phospholipids, and combinations of the foregoing.

In certain embodiments, the polymeric material is hydrophilic (e.g., PVA, PLLA, PLGA, PEG, and PAG). In certain other embodiments, the polymeric material is hydrophobic (e.g. silicone rubber, polyurethane, styrene-ethylene, butylene styrene, or styrene-isobutylene-styrene, etc).

More preferably for medical devices which undergo mechanical challenges, e.g. expansion and contraction, the polymeric materials should be selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition is capable of undergoing deformation under the yield point when the device is subjected to forces, stress or mechanical challenge.

In some embodiments, the polymeric materials are biodegradable. Biodegradable polymeric materials can degrade as a result of hydrolysis of the polymer chains into biologically acceptable, and progressively smaller compounds. In one embodiment, a polymeric material comprises polylactides, polyglycolides, or their co-polymers. Polylactides, polyglycolides, and their co-polymers break down to lactic acid and glycolic acid, which enters the Kreb's cycle and are further broken down into carbon dioxide and water.

The polymeric materials can also degrade through bulk hydrolysis, in which the polymer degrades in a fairly uniform manner throughout the matrix. For some novel degradable polymers, most notably the polyanhydrides and polyorthoesters, the degradation occurs only at the surface of the polymer, resulting in a release rate that is proportional to the surface area of the drug therapeutic agents and/or polymer/therapeutic agent mixtures. Hydrophilic polymeric materials such as PLGA will erode in a bulk fashion. Various commercially available PLGA may be used in the preparation of the coating compositions. For example, poly(d,l-lactic-co-glycolic acid) are commercially available. A preferred commercially available product is a 50:50 poly(d,l-lactic-co-glycolic acid) (d,l-PLA) having a mole percent composition of 50% lactide and 50% glycolide. Other suitable commercially available products are 65:35, 75:25, and 85:15 poly(d,l-lactic-co-glycolic acid). For example, poly(lactide-co-glycolides) are also commercially available from Boehringer Ingelheim (Germany) under the tradename Resomer®, e.g., PLGA 50:50 (Resomer RG 502), PLGA 75:25 (Resomer RG 752) and d,l-PLA (resomer RG 206), and from Birmingham Polymers (Birmingham, Ala.). These copolymers are available in a wide range of molecular weights and ratios of lactic to glycolic acid.

In one embodiment, the coating comprises copolymers with desirable hydrophilic/hydrophobic interactions (see, e.g., U.S. Pat. No. 6,007,845, which describes nanoparticles and microparticles of non-linear hydrophilic-hydrophobic multiblock copolymers, which is incorporated by reference herein in its entirety). In a specific embodiment, the coating comprises ABA triblock copolymers consisting of biodegradable A blocks from PLG and hydrophilic B blocks from PEO.

Furthermore, the term “therapeutic agent” as used in the present invention encompasses drugs, genetic materials, and biological materials and can be used interchangeably with “biologically active material”. Non-limiting examples of suitable therapeutic agent include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, taxol, paclitaxel, 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, 2′-O-ester with N-(dimethylaminoethyl)glutamide hydrochloride salt, nitroglycerin, nitrous oxides, nitric oxides, antibiotics, aspirins, digitalis, estrogen, estradiol and glycosides. In one embodiment, the therapeutic agent is a smooth muscle cell inhibitor or antibiotic. In a preferred embodiment, the therapeutic agent is taxol (e.g., Taxol®), or its analogs or derivatives. In another preferred embodiment, the therapeutic agent is paclitaxel, or its analogs or derivatives. In yet another preferred embodiment, the therapeutic agent is an antibiotic such as erythromycin, amphotericin, rapamycin, adriamycin, etc.

The term “genetic materials” means DNA or RNA, including, without limitation, of DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other non-genetic therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, rapamycin (sirolimus);
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds; and
macrolides such as sirolimus or everolimus.

Preferred biological materials include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, and 2′-O-ester with N-(dimethylaminoethyl)glutamide hydrochloride salt.

Other suitable therapeutic agents include tacrolimus; halofuginone; inhibitors of HSP90 heat shock proteins such as geldanamycin; microtubule stabilizing agents such as epothilone D; phosphodiesterase inhibitors such as cliostazole; Barkct inhibitors; phospholanban inhibitors; and Serca 2 genes/proteins.

Other preferred therapeutic agents include nitroglycerin, nitrous oxides, nitric oxides, aspirins, digitalis, estrogen derivatives such as estradiol and glycosides.

In one embodiment, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration.

In certain embodiments, the therapeutic agents for use in the medical devices of the present invention can be synthesized by methods well known to one skilled in the art. Alternatively, the therapeutic agents can be purchased from chemical and pharmaceutical companies.

Other embodiments could include electrodes on a biodegradable surface. Though the electrodes can remain after the degradation process, a biodegradable electrode can be developed using biodegradable electrolyte materials or a biodegradable electrode filled with biocompatible conductive particles, e.g., carbon, platinum, and/or titanium.

The stent 100 of FIG. 1 is shown in its unexpanded state. Certain of the struts 110 are configured in a sinuous manner or wave-like configuration. In this particular stent, the struts having a wave-like configuration can be connected to each other by struts that are relatively straight 115, i.e. longitudinal members. The struts having the wave-like configuration are capable of expanding in the radial direction. Generally, it is desirable to expand the stent when it is implanted into a body lumen so that the outer surface 120 of the stent contacts the surface of the body lumen or tubular organ. The inner surface of the stent (not shown), which is disposed opposite the outer surface, remains exposed to the fluid within the tubular organ and defines the lumenal space of the tubular organ.

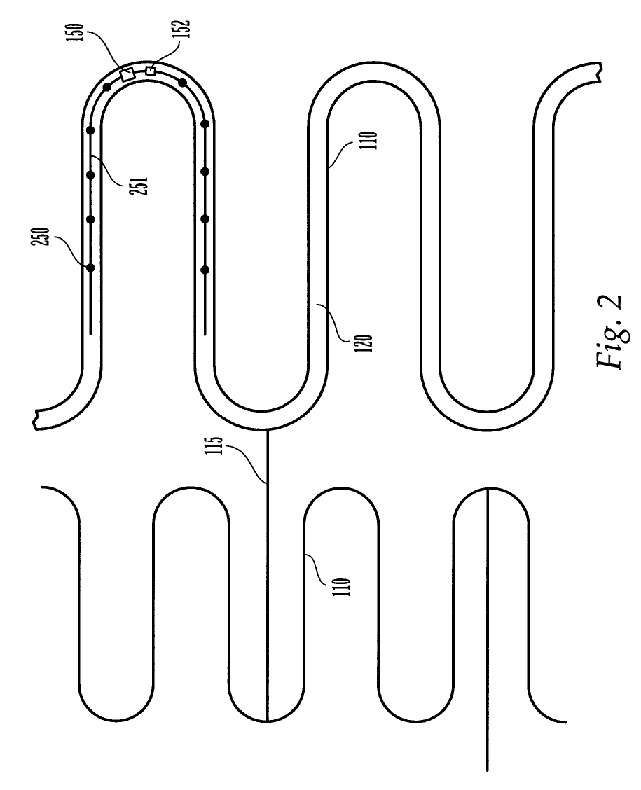

FIG. 2 is a magnified view of the device with electrodes 250 disposed on the outer surface 120 of the stent 100 shown in FIG. 1. In other embodiments, the electrodes can be disposed throughout the entire outer surface 120 or on other portions of the outer surface 120. Also, electrodes can be disposed on more than one portion of the outer surface 120. In addition, although the electrodes 250 in FIG. 2 are disposed on a portion of the outer surface 120, the electrodes 250 can be disposed instead or additionally on the inner surface of the stent 100. In some embodiments it is preferable to dispose the electrodes 250 on the outer surface 120 of the stent 100 because it is this surface that contacts the body lumen surface. In other embodiments, where biocompatibility with the fluid in the tubular organ or body lumen is desired, the electrode(s) is placed on the surface of the medical device that contacts the fluid. Also, preferably the electrode (250) are in electrical communication with each other. They may be electrically connected by the use of a connector 251 having the shape of a filament or other shape. One of skill in the art would be aware of suitable connectors.

Moreover, while the electrode 250 in FIG. 2 is depicted in the shape of circles or dots, the electrode can take on any suitable geometric configuration or shape. For example, the electrode can be configured as a band having a desired width. Also, the width of the electrode need not be uniform. Moreover, the electrode may be the same size as or smaller than cell receptors, e.g., less than 150 nm in length and/or width.

The electrode 250 may be made of biocompatible conducting material known to one of skill in the art, such as for example, aluminum, gold, or platinum. Sawyer, “Electrode-biologic tissue interactions at interfaces—A review.” Biomat., Med. Dev., Art. Org., 12(3-4), 161-196 (1984-85), herein incorporated by reference in its entirety, discloses the use of several electrode materials with respect to thrombogenic responses of the surrounding tissue that may be used to form electrodes. Also, metals known for exceptional biocompatibility, e.g., titanium, tantalum, tungsten, can be used as well as conductive polymers and polyelectrolyte hydrogels. In addition, noble metals may also be suitable materials.

The electrode 250 can be attached or connected to the surface of the device by using any of the micro-fabrication techniques known to one of skill in the art of semiconductor processing. Also, nanolithography, which is similar to microlithography, or microfabrication techniques can be used. However, nanolithography uses lasers of finer resolution/beam. Such techniques are described in Champagne et al., “Nanometer-scale scanning sensors fabricated using stencil lithography,” Applied Physics Letters, vol. 82, no. 7, Feb. 17, 2003. It should be noted that such techniques still require that wires and connectors be put in place.

In order to provide a current to the electrode, the medical device of the present invention comprises a power source 150 that is directly or indirectly in electrical communication with the electrode(s). The power source provides current to the electrode. Although the power source in FIG. 2 is depicted as being in direct physical contact with the electrode(s) 250, such direct physical contact with the electrode is not necessary. Suitable power sources for the present invention include, without limitation, implantable batteries, such as ones used with pacemakers, capacitors, and power sources comprising pick-up coils or induction coils. Traditional means of connecting the batteries to electrodes such as wires and circuit board-like connectors can be used. Also, Nems/Mems sensors could be prepared on the stent or in the battery and could be used to monitor, control and report through telemetry.

In one embodiment, the power source comprises an induction coil capable of being tuned to a preselected frequency. The induction coil can be in communication with a remote generator that is able to generate an oscillating magnetic field at the preselected frequency. The oscillating magnetic field is able to create a voltage across the induction coil to provide a source of power.

The power source can be attached to the medical device by various methods, such as welding or using an adhesive. Also, while the power source 150 is shown in FIG. 1 as being disposed on the same surface as the electrode, the power source can be disposed on or embedded in any surface of the strut or medical device. In addition, more than one power source may be used.

Optionally, the power source and electrode(s) are in electrical communication with a controller 152, which controls the current that is provided to the electrode(s) 250. The controller 152 may be attached or connected to the medical surface or may be fabricated directly onto the medical device using the methods known to one of skill in the art. For example, R. C. Jaeger, Introduction to Microelectronic Fabrication: Volume 5 of Modular Series on Solid State Devices, 2^{nd} ed., Prentice Hall (2001), herein incorporated by reference in its entirety, discloses the methods of microelectronic fabrication that may be adapted to fabricate the controller on the medical device. The controller may be disposed on the inner surface, the outer surface 120 of the medical device or other surface. Also the controller may be embedded in the device.

The controller 152 may include rectification, filtering, and voltage or current regulation circuits to create and maintain a desired or pre-selected current that is provided to the electrode. U.S. Pat. No. 5,279,292 issued to Baumann et al. and U.S. Pat. No. 6,327,504 issued to Dolgin et al., herein incorporated by reference in their entirety, disclose examples of such circuits that may be adapted by one of skill in the art without undue experimentation.

In certain embodiments, the power source is a pick-up coil that includes a conductor that forms at least one loop or turn and responds to an alternating magnetic field by creating a voltage potential difference between the two ends of the coil. The magnitude of the voltage potential depends in part on the number of turns in the coil, the area defined by the coil, the strength and orientation with respect to the coil area of the magnetic field crossing the coil area. In embodiments where the medical device is a stent, the surface normal to the coil area may be substantially parallel to the longitudinal axis of the stent. Alternatively, the surface of the coil normal may oriented away from the longitudinal axis of the stent in order, for example, to better align the coil to the alternating magnetic field.

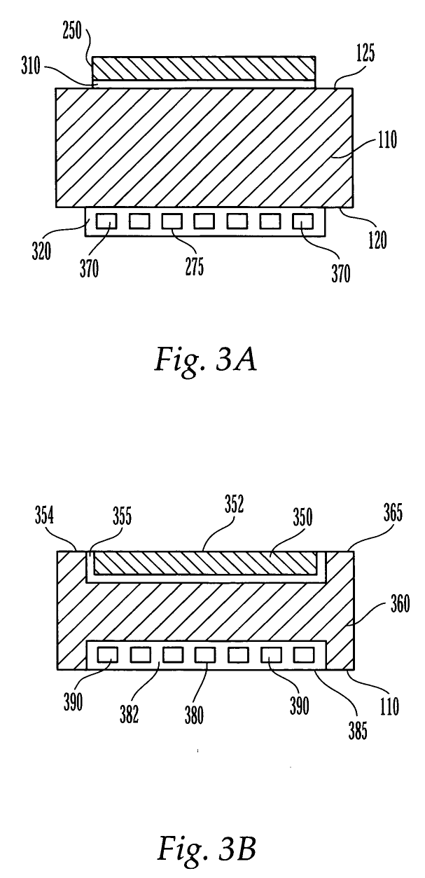

FIG. 3a depicts a sectional view of a stent strut 110 having an electrode 250 disposed on a surface of the strut. The electrode 250 is deposited on an insulating layer 310 that is deposited on a first surface 125 of the stent strut 110. The insulating layer 310 may be any biocompatible material that electrically insulates the electrode 250 from the stent strut 110 and exhibits good adhesion to the stent strut 110 and electrode 250. Insulating materials may include metal oxides or nitrides such as for example, silicon dioxide or silicon nitride, or polymers such as for example, polyimide, which is biocompatible when properly processed.

A pick-up coil 275 is imbedded in insulating material 320 to insulate the pick-up coil conductors 370 from each other, the stent strut 110, and from the host organism. Insulating material 320 may be the same material in insulating layer 310 or may be a different biocompatible insulating material. The pick-up coil 275 and insulating material 320 are disposed on the second surface 120 of the stent strut 110.

The pick-up coil 275 is in electrical communication with a controller (not shown) and is inductively coupled to an external coil (not shown). The induced voltage potential across the two ends of the pick-up coil 275 provides an externally generated power source to the controller. Alternatively, the medical device can comprise an additional internal power source, such that the induced voltage across the pick-up coil is used to recharge the internal power source.

The pick-up coil can be placed on any surface of the medical device. Also, the pick-up coil may be situated on a surface of the medical device that is the same or different from the surface upon which the electrode is disposed. The placement of the pick-up coil on or in the medical device is determined according to design and fabrication considerations such as, for example, medical device design, ease of fabrication or other factors known to one of skill in the art.

FIG. 3b provides a sectional view of an alternative embodiment in which the electrode and pick-up coil is embedded in the strut of a stent. In the embodiment shown in FIG. 3b, the exposed surface 352 of the electrode 350 is flush with a first surface 365 of a stent strut 360. The electrode 350 is insulated from the stent strut 360 by insulating material 355. The exposed surface 354 of the insulating material 355 is also flush with the first surface 365 of the stent strut 360. Like the embodiment shown in FIG. 3a, the embodiment shown in FIG. 3b includes a pick-up coil 380. In this embodiment, this pick-up coil is embedded in insulating material 382 to insulate the pick-up coil conductors 390 from each other, the stent strut 360, and from the host organism. Insulating material 382 may be the same as insulating material 355 or be a different biocompatible insulating material. The exposed surface 385 of the insulating material 382 is flush with the second surface 385 of the stent strut 360.

The embodiments shown in FIGS. 3a and 3b illustrate a single electrode having a width substantially the same as the width of the stent strut. Other embodiments, however, include more than one electrode disposed on the surface where active biocompatibility is desired. For example, electrodes having a width and electrode spacing in the range of 100-200 nm may be disposed on the stent surface. D. A. Rees et al., “Glycoproteins in the recognition of substratum by cultured fibroblasts,” Symp. Soc. Exp. Biol., 1978; 32:241-60, herein incorporated by reference in its entirety, discloses focal adhesions having uniform size in the 150 nm range. Disposing the electrodes to match the spacing observed in adhered cells may encourage adhesion.

As discussed above, by providing a current to the electrode disposed on a surface of a medical device, an average surface charge density is provided to or created on the surface. Such an average surface charge density provides the surface with biocompatibility properties. Preferably to promote biocompatibility, the average surface charge density should be negative. In particular, the electrodes can be used to change charge patterns at the level of receptors. The charge pattern can begin to replicate cell membrane charge patterns that result in cell interactions with the medical device surface that results in minimal activation of the cells to minimize inflammation. More specifically, as described by Helmus et al. and Thubrikar et al. (Thubrikar, M. et al., “Study of Surface Charge of the Intima and Artificial Materials in Relation to Thrombogenicity,” J. Biomech., vol. 13, pp. 663-666 (1980)), an average charge density that is similar to healthy endothelium imparts optimal thromboresistance. A surface having such an average charge density may mimic the sulfated glycosaminoglycans (“gags”), in particular heparin sulfate, that are an important component of cell membranes. The mimic of the negative surface charge of heparin sulfate not only produces a thromboresistant surface, but one that is highly biocompatible with respect to minimal activation of the inflammatory pathways. These types of electrodes may be of use in stimulation situations—nerve, skeletal muscle, heart muscle, other smooth muscle organs (e.g. GI tract), and neural.

Moreover, the cell interactions with the surface of the medical device can be controlled to encourage desired biological effects such as promoting cells to adhere and grow on the surface of the medical device. For instance, if the charge pattern replicates that of a natural surface, e.g. basement membrane rich in a cell adhesion peptide such as one composed of arginine-glycine-aspartic acid (RGD) so that the cells believe they are located on a compatible surface which encourages growth, the cells will grow. RGD is the peptide sequence in cell adhesive proteins such as fibronectin, laminin, etc. that is specific for cell receptors to attach.

The desired surface charge density may depend on the specific application of the medical device. Helmus et al. discloses a method of determining the desired surface charge density and is herein incorporated by reference in its entirety. Helmus et al. implanted random copolymers of L-glutamic and L-leucine into the femoral and carotid arteries of dogs and, determined that a negative surface charge density greater than about 5 μC/cm² was effective in reducing thrombus formation.

Also, an average surface charge density can be used to encourage thrombus formation for hemostatasis and tumor treatment or enhance inflammation or tissue formation. Preferably, in such embodiments, the average surface charge density comprises a net positive charge. The net positive charge can be created by the methods described above to create average surface charge density. A description of how the charged potential of metals result in lack or formation of thrombus is discussed in Srinivasan S. Sawyer P. N.; “Role of surface charge of the blood vessel wall, blood cells, and prosthetic materials in intravascular thrombosis,” J. Colloid Interface Sci. 1970 Mar:32(3):456-63, and Sawyer, P. N. and J. W. Pate, “Bioelectric Phenomena as an Etilogical Factor in Intravascular Thrombosis,” Amer. J. Physiol. 175:103 (1953), which are incorporated herein by reference in their entiety for all purposes.

Having thus described at least illustrative embodiments of the invention, various modifications and improvements will readily occur to those skilled in the art and are intended to be within the scope of the invention. Accordingly, the foregoing description is by way of example only and is not intended as limiting. The invention is limited only as defined in the following claims and the equivalents thereto.

All references mentioned herein are incorporated by reference in their entirety for all purposes.

What is claimed:

1. A medical device for implantation into a host organism, the device comprising:
a surface adapted for contact with body tissue of the host organism;
an electrode disposed on at least a portion of the surface; and
a power source in direct or indirect electrical communication with the electrode, wherein the power source is capable of providing a current to the electrode to create an average surface charge density on the surface that is effective to promote the biocompatibility of the surface with the body tissue.

2. The device of claim 1 wherein the average surface charge density comprises a net negative charge of positive and negative charges.

3. The device of claim 1 wherein the average surface charge density comprises a net positive charge of positive and negative charges.

4. The device of claim 1 wherein the electrode is less than about 150 nm in length or less than about 150 nm in width.

5. The device of claim 1 wherein the device comprises a substantially cylindrical shape, and wherein the surface defines a boundary of the cylindrical shape.

6. The device of claim 1 wherein the device is a stent.

7. The device of claim 1 wherein the power source comprises an induction coil, a battery or a pick-up coil

8. The device of claim 7, wherein the power source comprises an induction coil capable of being tuned to a preselected frequency.

9. The device of claim 8 wherein the induction coil is in communication with a remote generator capable of generating an oscillating magnetic field at the preselected frequency, wherein the oscillating magnetic field is capable of creating a voltage across the coil.

10. The device of claim 1 wherein the average surface charge density is maintained by a direct current.

11. The device of claim 1 wherein the average surface charge density is maintained by an alternating current.

12. The device of claim 11 wherein the alternating current is offset by a direct current base line.

13. The device of claim 1 wherein the average surface charge density is greater than 5 μC/cm².

14. The device of claim 1 wherein the average charge density ranges from about 0.05 to about 500 μC/cm².

15. The device of claim 1 wherein the average surface charge density ranges from about 0.5 to about 50 μC/cm².

16. A medical device for implantation into a host organism, the device comprising:
a first surface adapted for contact with a surface of a body lumen, wherein the body lumen contains a fluid;
a second surface adapted for contact with the fluid;
an electrode disposed on at least a portion of the first or second surface; and
a power source in direct or indirect electrical communication with the electrode, wherein the power source is capable of providing a current to electrode to create an average surface charge density on the first or second surface that is effective to promote the biocompatibility of the first or second surface with the surface of the body lumen or the fluid.

17. The device of claim 16 wherein the average surface charge density comprises a net negative charge of positive and negative charges.

18. The device of claim 16 wherein the average surface charge density comprises a net positive charge of positive and negative charges.

19. The device of claim 16 wherein the electrode is less than about 150 nm in length or less than about 150 nm in width.

20. The device of claim 16 further comprising a controller disposed on the first or second surface, wherein the controller is in electrical communication with the power source and the electrode and wherein the controller is capable of controlling the current provided to the electrode.

21. The device of claim 16 wherein the electrode is disposed on the first surface and the average surface charge density is created on the first surface to promote the biocompatibility of the first surface with the surface of the body lumen.

22. The device of claim 16 wherein the electrode is disposed on the second surface and the average surface charge density is created on the second surface to promote the biocompatibility of the second surface with the fluid.

23. The device of claim 16 wherein the device is a stent and the first surface is an outer surface of the stent and the second surface is an inner surface of the stent.

24. The device of claim 16 wherein the average surface charge density is greater than 5 μC/cm².

25. The device of claim 16 wherein the average surface charge density ranges from about 0.05 to about 500 μC/cm².

26. The device of claim 16 wherein the average surface charge density is from about 0.5 to about 50 μC/cm².

27. The device of claim 16 wherein the power source comprises a battery, a pick-up coil or an induction coil.

28. The device of claim 16 wherein the power source comprises a pick-up coil disposed on the first surface of the device.

29. The device of claim 28 wherein the pick-up coil is inductively coupled to a primary coil located external to the host organism.

30. A stent comprising:
a surface adapted for contact with body tissue of a host organism;
an electrode disposed on at least a portion of the surface; and
a power source comprising an induction coil in direct or indirect electrical communication with the electrode, wherein the induction coil is capable of providing a current to the electrode to create an average surface charge density on the surface that is effective to promote the biocompatibility of the surface with the body tissue, and wherein the average surface charge density is greater than 5 μC/cm² and wherein the average surface charge density comprises a net negative charge of positive and negative charges.

31. A medical device for implantation into a host organism, the device comprising:
a surface adapted for contact with body tissue of the host organism;
an electrode disposed on at least a portion of the surface; and
a power source in direct or indirect electrical communication with the electrode, wherein the power source is capable of providing a current to the electrode to create an average surface charge density on the surface that is effective to produce a desired biological effect.

32. The device of claim 31 wherein the biological effect is to encourage thrombus formation, enhance inflammation or enhance tissue formation.

33. The device of claim 31 wherein the enhancing tissue formation comprises enhancing fibrous tissue formation.

34. The device of claim 31 wherein the average surface charge density comprises a net positive charge of positive and negative charges.

35. The device of claim 31 wherein the electrode is less than about 150 nm in length or less than about 150 nm in width.

36. The device of claim 31 wherein the device comprises a substantially cylindrical shape, and wherein the surface defines a boundary of the cylindrical shape.

37. The device of claim 31 wherein the device is a stent.

38. The device of claim 31 wherein the power source comprises an induction coil, a battery or a pick-up coil

39. The device of claim 38 wherein the power source comprises an induction coil capable of being tuned to a preselected frequency.

40. The device of claim 39 wherein the induction coil is in communication with a remote generator capable of generating an oscillating magnetic field at the preselected frequency, wherein the oscillating magnetic field is capable of creating a voltage across the coil.

41. The device of claim 31 wherein the average surface charge density is maintained by a direct current.

42. The device of claim 31 wherein the average surface charge density is maintained by an alternating current.

43. The device of claim 31 wherein the alternating current is offset by a direct current base line.

44. The device of claim 31 wherein the average surface charge density is greater than 5 μC/cm².

45. The device of claim 31 wherein the average surface charge density ranges from about 0.05 to about 500 μC/cm².

46. The device of claim 31 wherein the average surface charge density ranges from about 0.5 to about 50 μC/cm².

47. A method of promoting the biocompatibility of a medical device for implantation into a host organism, comprising:
(a) obtaining a medical device having a surface adapted for contact with body tissue of the host organism;
(b) disposing an electrode on at least a portion of the surface; and
(c) disposing a power source in direct or indirect electrical communication with the electrode, wherein the power source is capable of providing a current to the electrode to create an average surface charge density on the surface that is effective to promote the biocompatibility of the surface with the body tissue.
